(19) United States
(12) Patent Application Publication
(10) Pub. No.: US 2010/0239643 A1
(43) Pub. Date: Sep. 23, 2010

(54) ANTI-ITCH SHEET

(76) Inventor: Shuhei Kurata, Osaka (JP)

Correspondence Address: WENDEROTH, LIND & PONACK, L.L.P., 1030 15th Street, N.W.,, Suite 400 East, Washington, DC, 20005-1503, US

(21) Appl. No.: 12/734,910

(22) PCT Filed: Dec. 26, 2008
(86) PCT No.: PCT/JP2008/073680

§ 371 Date: Jun. 2, 2010

(30) Foreign Application Priority Data
Dec. 28, 2007 (JP) 2007-340194

(51) Int. Cl. A61K 9/70; A61K 47/32; A61K 47/34; A61P 17/00

(52) U.S. Cl. 424/443; 514/772.4; 514/772.3

(57) ABSTRACT

The present invention provides an anti-itch sheet suitable for curing and relieving itching caused by an insect bite or sunburn based on a novel finding, the anti-itch sheet comprising a sealing layer, and a fixing layer on a skin-contacting side of the sealing layer for facilitating the application of the sealing layer to the skin.

### TECHNICAL FIELD

The present invention relates to provision of a useful sticking sheet for curing or relieving itching. In particular, the present invention provides a useful sticking sheet for quickly curing or relieving unbearable itching caused by an insect bite especially from a mosquito, a hairy caterpillar, a black fly, or the like, or itching caused by sunburn.

### BACKGROUND ART

Currently, commercially available anti-itch medicines are provided in various forms such as liquid medicines and ointment medicines. Each anti-itch medicine contains an antipruritic for relieving itching or an anti-inflammatory for reducing inflammation, and sometimes further contains, along with these medicines, an anesthetic for topically paralyzing nerves in order for itching not to be felt. Known examples of the antipruritic or the anti-inflammatory include an antihistamine such as diphenhydramine hydrochloride, and a steroid such as dexamethasone acetate. Known examples of the anesthetic include procaine, tetracaine, dibucaine, lidocaine. Also, an anti-itch medicine with a further added auxiliary agent such as L-menthol and camphor is known.

Furthermore, a topically applicable anti-itch preparation comprising a centella asiatica extract and tea tree oil is disclosed in WO 2003/47609.

JP-A-2004-161642 also discloses that a patch comprising a metal ion such as Ag, Mn, Fe, Co, Ni, Cu, and Zn is effective for stopping itching.

The added/contained medicine or metal ion in each of the anti-itch medicines mentioned above is intended to exert an antipruritic effect.

### SUMMARY OF INVENTION

### Technical Problem

The present invention provides an anti-itch sheet that exerts an effect of stopping or relieving itching caused by an insect bite from a mosquito, a hairy caterpillar, a black fly, or the like, or caused by sunburn.

### Solution to Problem

The present inventors made extensive investigations on itching especially caused by an insect bite from a mosquito, a hairy caterpillar, a black fly, or the like, or caused by sunburn, and found that itching was not felt when an insect bitten area or a sunburned area was not exposed to the air; thus, the inventors have completed the present invention. That is, the present inventors got an unexpected novel finding that itching can be stopped by immediate sealing of an insect bitten area or a sunburned area to shut out the air, and used considerable ingenuity to put the finding to practical use. As a result, the inventors succeeded in developing a method for practical use thereof, and have completed the present invention.

Namely, the present invention relates to

(1) an anti-itch sheet comprising a sealing layer, and a fixing layer on a skin-contacting side of the sealing layer;

(2) the anti-itch sheet according to the above-mentioned (1), wherein the sealing layer is a gel layer and the fixing layer is an adhesive layer;

(3) the anti-itch sheet according to the above-mentioned (1) or (2), wherein the fixing layer has the sealing layer on a non-skin-contacting side of the fixing layer, and a protective sheet for the fixing layer on a skin-contacting side of the fixing layer;

(4) the anti-itch sheet according to the above-mentioned (3), wherein the sealing layer comprises a reinforcement;

(5) the anti-itch sheet according to the above-mentioned (3), wherein the sealing layer comprises one or more selected from a silicone resin, a urethane resin, and an acrylic resin;

(6) the anti-itch sheet according to the above-mentioned (3), wherein the fixing layer comprises one or more selected from an acrylic adhesive, a rubber adhesive, and a urethane adhesive;

(7) the anti-itch sheet according to the above-mentioned (3), wherein the fixing layer contains an antipruritic;

(8) the anti-itch sheet according to the above-mentioned (7), wherein the antipruritic is an antihistamine and/or a steroid;

(9) an anti-itch sheet comprising a sealing layer and a reinforcement thereof;

(10) the anti-itch sheet according to the above-mentioned (9), wherein the sealing layer contains an antipruritic;

(11) a therapeutic substance for itching caused by an insect bite or sunburn, comprising the anti-itch sheet according to the above-mentioned (1);

(12) a use of the anti-itch sheet for producing the therapeutic substance for itching caused by an insect bite or sunburn according to the above-mentioned (11); and

(13) a medical treatment for itching caused by an insect bite or sunburn, comprising adhering the anti-itch sheet according to the above-mentioned (1) to an itching area in a sealing manner.

### ADVANTAGEOUS EFFECTS OF INVENTION

The anti-itch sheet provided by the present invention is capable of effectively curing or relieving itching caused by an insect bite or sunburn even without an antipruritic, which is contained in a conventional anti-itch medicine. Therefore, the anti-itch sheet can be used without concern about overresponse to the medicine such as a rash and hives, or wearing-off of the effect due to deterioration of the medicine.

### BRIEF DESCRIPTION OF DRAWINGS

FIG. 1 is a schematic view (sectional view) of the anti-itch sheet of the present invention.

FIG. 2 is a schematic view (sectional view) of the anti-itch sheet, comprising a sealing layer having adhesiveness on a skin-contacting side.

FIG. 3 is a schematic view (sectional view) of the anti-itch sheet, wherein a sealing layer has adhesiveness on both a non-skin-contacting side and a skin-contacting side, and the sealing layer is provided with a tack-blocking sheet on the non-skin-contacting side.

FIG. 4 is a schematic view (sectional view) of the anti-itch sheet, wherein a sealing layer has adhesiveness on a skin-contacting side, and the sealing layer is stacked with a reinforcement on a non-skin-contacting side.

FIG. 5 is a schematic view (sectional view) of the anti-itch sheet comprising three layers of a sealing layer, a fixing layer, and a reinforcement, wherein a non-skin-contacting side of the sealing layer is stacked with a reinforcement.

FIG. 6 is a schematic view (sectional view) of the anti-itch sheet, wherein a sealing layer has adhesiveness on both a non-skin-contacting side and a skin-contacting side, and the sealing layer is provided with a tack-blocking sheet on the non-skin-contacting side and a reinforcement in the sealing layer.

FIG. 7 is a schematic view (sectional view) of the anti-itch sheet having a reinforcement in a sealing layer.

FIG. 8 is a schematic view (sectional view) of the anti-itch sheet, wherein a sealing layer is provided with a peelable reinforcement on a non-skin-contacting side; and a schematic view (sectional view) of the anti-itch sheet showing that it is possible to peel off only the reinforcement after the anti-itch sheet is put on an itching area.

### REFERENCE SIGNS LIST

1. Sealing layer
2. Fixing layer
3. Sealing layer having adhesiveness on a skin-contacting side
4. Sealing layer having adhesiveness on both a non-skin-contacting side and a skin-contacting side
5. Tack-blocking sheet
6. Reinforcement
7. Itching area

### DESCRIPTION OF EMBODIMENTS

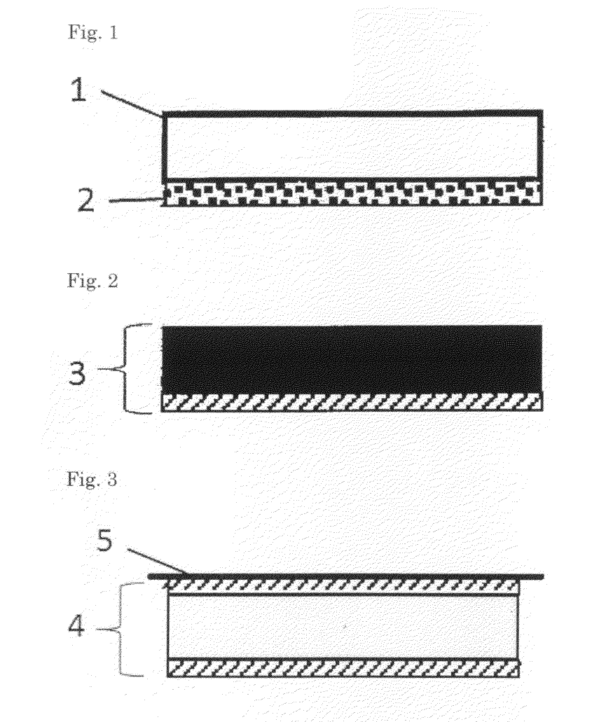

The anti-itch sheet provided by the present invention has a structure described below. As shown in FIG. 1, the anti-itch sheet has in principle a structure comprising a sealing layer, and a fixing layer on a skin-contacting side of the sealing layer. The sealing layer refers to a sheet that is intended to cutoff contact with the air, and composed of a raw material having a capability of shutting out the air. The capability of shutting out the air includes, besides the capability of completely cutting off contact with the air, a limited capability of substantially cutting off contact between an insect bitten area and the air. The fixing layer refers to what is necessary for fixing the sealing layer on a certain insect bitten area, composed of a raw material having adhesiveness. The sealing layer may be sometimes referred to as an air-shutout layer in the present invention. The following is the same as above.

The anti-itch sheet provided by the present invention comprises the structure having the requirements mentioned above. Furthermore, the anti-itch sheet is suitably provided with usual modifications to facilitate the production, storage, carry, and use, and further to enhance its effect. The usual modifications are as follows:

(A) providing a protective sheet on a skin-contacting side of the fixing layer to protect an adhesive layer;
(B) providing a reinforcement on a non-skin-contacting side of the sealing layer if necessary to support the sealing layer, or to increase the stiffness of the sealing layer; and
(C) impregnating a medicine having efficacy for relieving itching in the fixing layer.
All these mentioned above are included in the present invention.

Suitable examples of the above-mentioned sealing layer include a gel layer comprising a plastic sheet. The plastic sheet can be produced from a plastic material singly or in a combination of two or more kinds. Plastic sheets thus obtained may be stacked up and used as a layered material. Examples of the plastic material include a polymer such as polyethylene, polypropylene, polyester, polyvinylidene chloride, and polyamide; an acrylic resin-based gelled substance prepared from a polymer obtained by polymerizing acrylic acid, methacrylic acid, and an ester thereof in a suitable combination, for example, a resin-based gelled substance obtained by polymerizing alkoxy ethyl(meta)acrylate and a divinyl compound such as N,N′-methylenebisacrylamide, ethylene glycol methacrylate, and the like with the use of azobisisobutyronitrile, benzoyl peroxide, or the like; a urethane resin-based gelled substance, for example, a resin-based gelled substance obtained by polymerizing polyether polyol and toluene diisocyanate with the use of a plasticizer in the presence of dibutyltin laurate as a catalyst; and a silicone resin-based gelled substance, for example, a resin-based gelled substance obtained from a gelled substance of alkyl polysiloxane. The plastic material for the sealing layer is preferably one or more selected from the silicone resin-based gelled substance, the urethane resin-based gelled substance, and the acrylic resin-based gelled substance. Among these gelled substances, the silicone-based gelled substance is especially preferable. Alternatively, the sealing layer may be a film made of a polyolefin such as polyethylene and polypropylene; polyurethane; polyamide; polyester; polyvinyl chloride; polyvinylidene chloride; or the like. The thickness of the sealing layer is usually 5 μm to 1 mm, preferably 10 to 150 μm. This is because a sheet less than 5 μm thick cannot keep enough strength, and a sheet more than 1 mm thick is too thick and bulky. The sealing layer may have a vapor permeability not to trap excessive moisture by insensible perspiration. The vapor permeability is 300 to 12,000 g/m²·24 hours.

Examples of the above-mentioned gelled substance of alkyl polysiloxane include a gelled substance produced according to a disclosure such as JP-A-62-20557, JP-A-62-181357, and JP-A-2000-26733. Suitable examples of the alkyl polysiloxane include alkyl polysiloxane obtained by mixing (a) organopolysiloxane having two or more vinyl groups at the molecular ends or in the molecule, and (b) organopolysiloxane having a hydrogen atom directly bonded to silicon in the molecule, and depending on the purpose, subsequently crosslinking/hardening the mixture in the presence of a platinum catalyst, with heating or the like if necessary.

Examples of (a) organopolysiloxane having vinyl groups at the molecular ends include vinylmethyl-dimethylpolysiloxane, vinylmethyl-phenylmethyl-dimethylpolysiloxane. Examples of (b) organopolysiloxane having a hydrogen atom directly bonded to silicon in the molecule include methylhydrogensiloxane-dimethylsiloxane cyclic copolymer, dimethylsiloxane-methylhydrogensiloxane copolymer terminated at both molecular ends with trimethylsiloxy groups.

With the use of the above-mentioned organopolysiloxane mixture before hardened, a sheet-shaped, silicone resin-based gelled substance to be used as the sealing layer can be produced by applying the organopolysiloxane mixture to a peelable base material, for example, a film made of a fluorine resin, polypropylene, polyethylene, polyester, polyethylene terephthalate, or the like; a sheet of paper on which such a film is pasted; kraft paper treated with fluorosilicone; or the like with the use of an apparatus such as a doctor knife, a roll coater, and a wire coater, and subsequently hardening the organopolysiloxane mixture with heating or the like. The acrylic resin-based gelled substance and the urethane resin-based gelled substance can also be produced by the same method.

The above-mentioned fixing layer is provided on the skin-contacting side of the sealing layer to function to fix the sealing layer on the skin. The fixing layer is preferably an adhesive layer comprising an adhesive.

The adhesive used for the adhesive layer is preferably one or more selected from an acrylic adhesive, a rubber adhesive, and a urethane adhesive. Examples of the acrylic adhesive include a polymer known per se obtained by polymerizing or copolymerizing one or more monomers selected from acrylic acid, methacrylic acid, and an ester thereof, but not particularly limited thereto. Examples of the monomer include a monomer known per se such as acrylic acid methyl ester, acrylic acid ethyl ester, acrylic acid (iso- or n-)propyl ester, acrylic acid (iso- or n-)butyl ester, acrylic acid 2-ethylhexyl ester, acrylic acid octyl ester, acrylic acid pentyl ester, acrylic acid heptyl ester, acrylic acid amide, methacrylic acid methyl ester, methacrylic acid ethyl ester, methacrylic acid (iso- or n-)propyl ester, methacrylic acid (iso- or n-)butyl ester, methacrylic acid octyl ester, methacrylic acid pentyl ester, methacrylic acid heptyl ester, and methacrylic acid amide.

Examples of the rubber adhesive include a rubber adhesive known per se such as natural rubber, synthetic rubber, styrene-butadiene rubber, isoprene rubber, styrene-butadiene-styrene block copolymer, styrene-isoprene-styrene block copolymer, polyisobutylene, and styrene-butadiene rubber, but not particularly limited thereto.

Examples of a silicone adhesive include a silicone adhesive known per se such as dialkyl siloxane polymer, for example, dimethylsiloxane polymer and diethylsiloxane polymer, but not particularly limited thereto.

These adhesives can be used, regardless of their kinds, singly or in a combination thereof. When the adhesives are mixed, for example, polyvinylpyrrolidone can also be added thereto to increase the adhesiveness of the adhesive layer. The adhesive layer can be provided on the skin-contacting side of the above-mentioned sealing layer by applying the adhesive according to a conventional method. The thickness of the fixing layer is usually 5 to 100 μm, preferably 10 to 30 μm. The adhesive power of the fixing layer to a steel board is usually about 0.5 to 8.0 N/cm, preferably about 2.0 to 4.0 N/cm. This is because an adhesive power less than 0.5 N/cm is not enough to fix the anti-itch sheet, and an adhesive power more than 8.0 N/cm is too strong to peel the sheet off after use.

In cases where the sealing layer itself has enough adhesiveness (tackiness) to adhere to the skin, the anti-itch sheet can be directly fixed on the skin only with the sealing layer; thus, the anti-itch sheet of the present invention does not need to be stacked with a fixing layer. Examples of the sealing layer having such adhesiveness include a sealing layer having adhesiveness on a skin-contacting side as shown in FIG. 2 (hereinafter, abbreviated as a sealing layer having single-sided tackiness), and a sealing layer having adhesiveness on both a non-skin-contacting side and a skin-contacting side as shown in FIG. 3 (hereinafter, abbreviated as a sealing layer having double-sided tackiness). Specifically, suitable examples of the sealing layer having double-sided tackiness include a gelling sheet. The gelling sheet can be produced from, singly or in a combination of two or more kinds, a high-polymer material or the like that responses to the temperature of a living body when applied thereto, turns from a solution to a gel, and forms a gel film on the application area. The obtained gelling sheet can also be used as a layered material. Suitable examples of the high-polymer material include a temperature-responsive material (or a temperature-responsive polymer) disclosed in JP-A-2003-252936. In cases where a fixing layer is not needed as mentioned above, an anti-itch sheet comprising a tack-blocking sheet (for example, a film, nonwoven fabric, paper, a gel sheet, or the like) stacked on a non-skin-contacting side of the sealing layer, and a peelable protective sheet directly provided on a skin-contacting side of the sealing layer can also be produced. The protective sheet will be suitably selected from protective sheets described below, and used.

A peelable protective sheet for the fixing layer is preferably provided on the skin-contacting side of the fixing layer of the anti-itch sheet of the present invention in view of the production or the storage. The protective sheet is made of a thin film made of polyester, polypropylene, polyethylene, polyvinylidene chloride, polyamide, a fluorine resin, or the like, or paper coated with these resins. The surface of the film or the paper is treated with a silicone compound such as fluorosilicone, which allows the protective sheet to be peeled off easily.

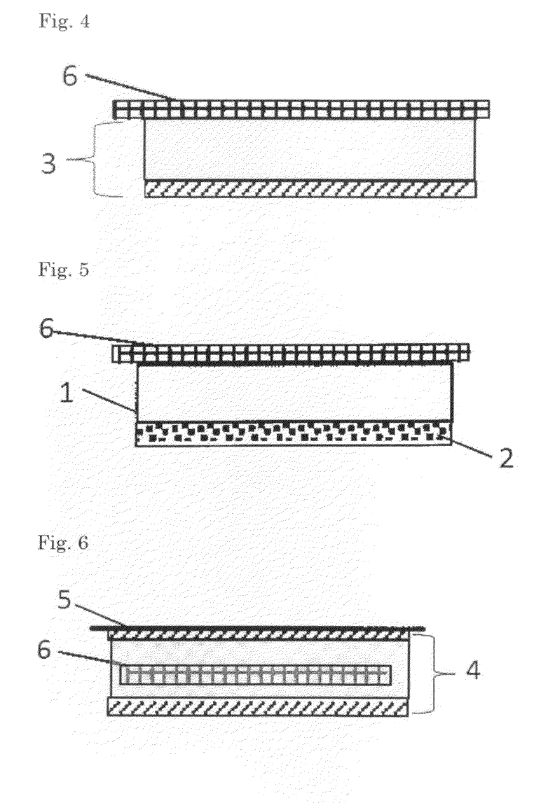

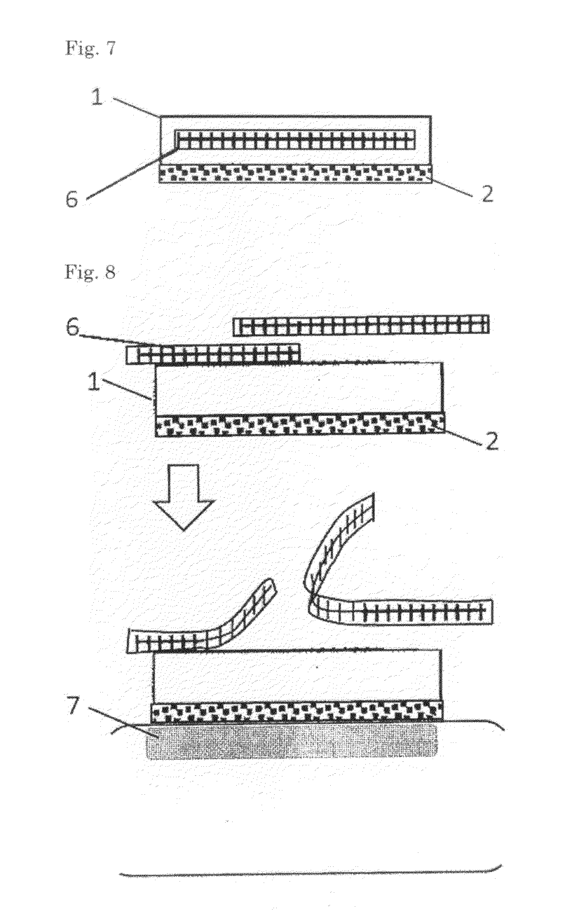

A reinforcement may be provided for the anti-itch sheet of the present invention, if necessary, to increase the stiffness of the sealing layer or to support the sealing layer. The reinforcement not only reinforces the sealing layer but also makes putting on the anti-itch sheet easier. Examples of the reinforcement include, besides the above-mentioned tack-blocking sheet provided on the sealing layer, a material known per se such as a plastic sheet obtained by using a polymer, for example, polyethylene, polypropylene, polyester, polyvinylidene chloride, polyamide, ethylene vinyl acetate, or the like, singly or in a combination thereof; a silicone sheet; a FRP sheet; a film material made of polyethylene, polypropylene, polyester, silicone, or the like; woven or knitted fabric; paper (for example, release paper or the like); and cloth. The reinforcement is preferably removable after the anti-itch sheet is put on. The reinforcement may be provided on the non-skin-contacting side of the sealing layer as shown in FIG. 4 and FIG. 5, or alternatively sandwiched by sealing layers. In cases where the reinforcement is provided on the non-skin-contacting side of the sealing layer, the reinforcement may be peelably provided on the non-skin-contacting side of the sealing layer as shown in FIG. 8. With such a peelable reinforcement, the anti-itch sheet can be easily put on even when the anti-itch sheet is a thin film. After the sheet is put on, discomfort felt during the putting can be eliminated by peeling only the reinforcement off the sheet. In the embodiment shown in FIG. 4, the sealing layer has adhesiveness on the skin-contacting side and functions also as a fixing layer; therefore, an additional adhesive layer is not necessarily provided as a fixing layer. Alternatively, the reinforcement may be provided in the sealing layer as shown in FIG. 6 and FIG. 7. The reinforcement reinforces the anti-itch sheet, especially the sealing layer, and makes putting on and peeling off easier.

The anti-itch sheet of the present invention may contain an antipruritic in the fixing layer. Suitable examples of the antipruritic contained in the fixing layer include an antihistamine and/or a steroid, but not particularly limited thereto. Examples of the antihistamine include diphenhydramine hydrochloride, d-chlorpheniramine maleate, and mequitazine. Examples of the steroid include methylprednisolone acetate, dexamethasone acetate, dexamethasone propionate, prednisolone acetate, prednisolone valerate acetate, and hydrocortisone acetate. The content of these medicines varies depending on the kind thereof. The content of the antihistamine is adjusted to about 1% by weight or less to the total weight of the anti-itch sheet. The content of the steroid is adjusted to about 0.03% by weight or less to the total weight of the anti-itch sheet. In addition to these medicines, a medicine that gives refreshingness and exerts a preservative/disinfectant effect during the use of the anti-itch sheet of the present invention, for example, menthol, isopropylmethylphenol, camphor, and the like can be added if necessary.

It is satisfactory for the anti-itch sheet of the present invention to have a strength only enough to shut out the air during the application to the skin, and enough not to break when peeled off after use; however, considering residues left on the skin, the anti-itch sheet of the present invention preferably has a suitable thickness to prevent a break or difficult peeling that may occur when the sheet is peeled off for disposal after use. The combined thickness of the sealing layer and the fixing layer of the anti-itch sheet is usually about 0.01 to 1.1 mm, preferably about 0.02 to 0.18 mm.

The size of the anti-itch sheet of the present invention is not particularly limited, but when the sheet is used for curing itching caused by an insect bite, the size is preferably enough to completely cover a swelling caused by the insect bite, for example, about 30 mm×30 mm or more.

### EXAMPLES

The present invention will be specifically described by the following Examples and Comparative Example, but not limited thereto.

### Example 1

An anti-itch sheet was produced by the following process with the use of a polyurethane film as a sealing layer, a polypropylene film as a reinforcement, an acrylic adhesive as a fixing layer, and silicone coated paper as a protective sheet.

The one side of a polyurethane film 30 μm thick was reinforced with a polypropylene film. An acrylic adhesive was applied to the back of the polyurethane film so as to be 20 μm in the adhesive thickness. The adhesive power of the fixing layer was 2.2 N/cm (to a steel board). Next, the adhesive was covered with a silicone coated paper, and thus a layered sheet was produced. The layered sheet was punched out into anti-itch sheets each 30 mm in length and width.

For use, the release paper is removed, and then the anti-itch sheet is put on so as for the exposed adhesive layer to cover an itching area in a fixing manner. After covering, by peeling off the reinforcement, the affected area having itching can be covered only with the thin film of the sealing layer and the adhesive layer.

### Comparative Example 1

A commercially available antipruritic and anti-inflammatory agent (an antihistamine, product name: Muhi S produced by Ikeda Mohando Co., Ltd.) was used for Comparative Example 1.

### Test Example 1

The forearm of a subject was inserted for several minutes in a sterilized box containing seven or eight female culex pipiens mosquitoes. From at least three spots on the forearm, the blood was sucked by the culex pipiens mosquitoes. Several minutes later, after the itching areas swelled, the following treatments were given.

### Treatment Example 1

The anti-itch sheet obtained in Example 1 was put on the insect bitten area.

### Treatment Example 2

A suitable amount of the antipruritic and anti-inflammatory agent for Comparative Example 1 was applied (topically applied) to the blood-sucked area.

Control: No treatment was given after the blood was sucked.

The test was carried out on ten subjects. The degrees of itching and its relieving five minutes after the treatment were evaluated by averaging scores given by the subjects based on their sensory perception according to the following evaluation criteria having four grades. The results are shown in Table 1.

Evaluation criteria: feel no itching (3 points), feel weak itching (2 points), feel itching (1 point), feel strong itching (0 point).

### Results

TABLE 1

| | Treatment | Treatment | |
|---|---|---|---|
| | Example 1 | Example 2 | Control |
| Average of | 2.5 | 1.9 | 0.0 |
| Evaluation Points | | | |

### INDUSTRIAL APPLICABILITY

The anti-itch sheet provided by the present invention is used for curing or relieving itching caused by an insect bite or sunburn; therefore, it is expected that the anti-itch sheet will be used in the same manner as an anti-itch medicine in the medical field.

1. An anti-itch sheet comprising a sealing layer, and a fixing layer on a skin-contacting side of the sealing layer.

2. The anti-itch sheet according to claim 1, wherein the sealing layer is a gel layer and the fixing layer is an adhesive layer.

3. The anti-itch sheet according to claim 1, wherein the fixing layer has the sealing layer on a non-skin-contacting side of the fixing layer and a protective sheet for the fixing layer on a skin-contacting side of the fixing layer.

4. The anti-itch sheet according to claim 3, wherein the sealing layer comprises a reinforcement.

5. The anti-itch sheet according to claim 3, wherein the sealing layer comprises one or more selected from a silicone resin, a urethane resin, and an acrylic resin.

6. The anti-itch sheet according to claim 3, wherein the fixing layer comprises one or more selected from an acrylic adhesive, a rubber adhesive, and a urethane adhesive.

7. The anti-itch sheet according to claim 2, wherein the fixing layer has the sealing layer on a non-skin-contacting side of the fixing layer and a protective sheet for the fixing layer on a skin-contacting side of the fixing layer.
